(19) United States
(12) Patent Application Publication
(10) Pub. No.: US 2026/0207049 A1
(43) Pub. Date: Jul. 23, 2026

(54) OPTICAL BIOMETER AND OPERATING METHOD THEREOF

(71) Applicant: CRYSTALVUE MEDICAL CORPORATION, Taoyuan City (TW)

(72) Inventors: CHUN-NAN LIN, Taoyuan City (TW); Yen-Jen Chang, Taoyuan City (TW); Tung-Yu Lee, Taoyuan City (TW); Che-Liang Tsai, Taoyuan City (TW); Hsuan-Hao Chao, Taoyuan City (TW); William Wang, Taoyuan City (TW); Chung-Ping Chuang, Taoyuan City (TW)

(21) Appl. No.: 19/450,871

(22) Filed: Jan. 16, 2026

Related U.S. Application Data
(60) Provisional application No. 63/748,747, filed on Jan. 23, 2025.

(51) Int. Cl. A61B 3/10

(52) U.S. Cl. CPC A61B 3/102

(57) ABSTRACT

An optical biometer is disclosed. In the optical biometer, an optical coupling module receives an incident light from a light source module and emits a reference light and a sensing light; a switchable reference arm reflects the reference light to generate a first reflected light; the sensing light is reflected by a specific interface of an eye to generate a second reflected light; the optical coupling module causes the first and second reflected lights to interfere to generate an optical interference signal; the detection module generates a detection result related to the eye based on the optical interference signal. When the reference light emitted into the switchable reference-arm does not fall on a preset reference-arm position corresponding to the specific interface of eye, an optical path of reference light is adjusted by a translation mechanism along an optical axis, so the reference light falls on the preset reference-arm position.

### CROSS REFERENCE TO RELATED APPLICATION

This application claims priority to and the benefit of U.S. application Ser. No. 63/748,747, having a filing date of Jan. 23, 2025, the entire content of which is incorporated herein by reference as if fully set forth below in its entirety and for all applicable purposes.

### TECHNICAL FIELD

The invention relates to a biometer, more particularly, to an optical biometer and an operating method thereof.

### BACKGROUND

In general, conventional optical coherence tomography (OCT) biometers usually have unavoidable manufacturing tolerances, such as those related to characteristic parameters of optical components, manufacturing tolerances of mechanical parts, and cutting tolerances of optical coupling modules. These tolerances will cause the reference light to deviate from its intended position on the reference arm, resulting in poor measurement accuracy.

In conventional designs, these manufacturing tolerances can be overcome by increasing the drive stroke of motor screws. However, the length of the motor screw is usually limited by space constraints; therefore, it is necessary to repeatedly trim the length of the optical coupling module and discard optical coupling modules with excessive length errors, but this also increases production costs and needs to be improved.

### SUMMARY

In view of this, an optical biometer and an operating method thereof are proposed in the invention to effectively solve the above-mentioned problems in the prior art.

An embodiment of the invention is an optical biometer. In this embodiment, the optical biometer includes a light source module, an optical coupling module, a switchable reference arm and a detection module. The light source module is configured to emit an incident light. The optical coupling module is disposed corresponding to the light source module and configured to receive the incident light and emit a reference light and a sensing light respectively. The switchable reference arm is disposed corresponding to the optical coupling module and configured to reflect the reference light to generate a first reflected light to the optical coupling module. When the sensing light is emitted to a specific interface of an eye under test, the specific interface of the eye under test reflects the sensing light to generate a second reflected light to the optical coupling module. The optical coupling module causes the first reflected light and the second reflected light to interfere to generate an optical interference signal. The detection module is disposed corresponding to the optical coupling module and configured to generate a detection result related to the eye under test according to the optical interference signal. When the reference light emitted from the optical coupling module into the switchable reference arm does not fall on a preset reference-arm position corresponding to the specific interface of the eye under test, an optical path of the reference light is adjusted by a translation mechanism along an optical-axis, so that the reference light can fall on the preset reference-arm position.

In an embodiment, when the optical biometer operates in a retina mode, the specific interface of the eye under test is retina; when the optical biometer operates in a cornea mode, the specific interface of the eye under test is cornea.

In an embodiment, the translation mechanism includes adjusting a position of the optical coupling module, the switchable reference arm, or the light source module along the optical axis.

In an embodiment, an adjustment performed on the optical path of the reference light is a stepped adjustment or a stepless adjustment.

Another embodiment of the invention is an optical biometer. In this embodiment, the optical biometer includes a light source module, an optical coupling module, a switchable reference arm, a detection module and a light-transmitting medium. The light source module is configured to emit an incident light. The optical coupling module is disposed corresponding to the light source module and configured to receive the incident light and emit a reference light and a sensing light respectively. The switchable reference arm is disposed corresponding to the optical coupling module and configured to reflect the reference light to generate a first reflected light to the optical coupling module. When the sensing light is emitted to a specific interface of an eye under test, the specific interface of the eye under test reflects the sensing light to generate a second reflected light to the optical coupling module. The optical coupling module causes the first reflected light and the second reflected light to interfere to generate an optical interference signal. The detection module is disposed corresponding to the optical coupling module and configured to generate a detection result related to the eye under test according to the optical interference signal. The light-transmitting medium is disposed between the optical coupling module and the switchable reference arm. When the reference light emitted from the optical coupling module into the switchable reference arm does not fall on a preset reference-arm position corresponding to the specific interface of the eye under test, an optical path of the reference light is adjusted by a translation mechanism along an optical axis, so that the reference light can fall on the preset reference-arm position.

Another embodiment of the invention is a method for operating an optical biometer. In this embodiment, the optical biometer includes a light source module, an optical coupling module, a switchable reference arm and a detection module. The method includes steps of: the light source module emitting an incident light; the optical coupling module disposed corresponding to the light source module receiving the incident light and emitting a reference light and a sensing light respectively; the switchable reference arm disposed corresponding to the optical coupling module reflecting the reference light to generate a first reflected light to the optical coupling module; the sensing light being emitted to a specific interface of an eye under test, the specific interface of the eye under test reflecting the sensing light to generate a second reflected light to the optical coupling module; the optical coupling module causing the first reflected light and the second reflected light to interfere to generate an optical interference signal; the detection module disposed corresponding to the optical coupling module generating a detection result related to the eye under test according to the optical interference signal; and when the reference light emitted from the optical coupling module into the switchable reference arm does not fall on a preset reference-arm position corresponding to the specific interface of the eye under test, an optical path of the reference light is adjusted by a translation mechanism along an optical-axis, so that the reference light can fall on the preset reference-arm position.

In an embodiment, when the optical biometer operates in a retina mode, the specific interface of the eye under test is retina; when the optical biometer operates in a cornea mode, the specific interface of the eye under test is cornea.

In an embodiment, the translation mechanism include adjusting a position of the optical coupling module, the switchable reference arm, or the light source module along the optical axis.

In an embodiment, the adjustment of the optical path of the reference light is a stepped adjustment or a stepless adjustment.

Another embodiment of the invention is a method for operating an optical biometer. In this embodiment, the optical biometer includes a light source module, an optical coupling module, a switchable reference arm and a detection module. The method includes steps of: the light source module emitting an incident light; the optical coupling module disposed corresponding to the light source module receiving the incident light and emitting a reference light and a sensing light respectively; the switchable reference arm disposed corresponding to the optical coupling module reflecting the reference light to generate a first reflected light to the optical coupling module; the sensing light being emitted to a specific interface of an eye under test, the specific interface of the eye under test reflecting the sensing light to generate a second reflected light to the optical coupling module; the optical coupling module causing the first reflected light and the second reflected light to interfere to generate an optical interference signal; the detection module disposed corresponding to the optical coupling module generating a detection result related to the eye under test according to the optical interference signal; disposing a light-transmitting medium between the optical coupling module and the switchable reference arm; and when the reference light emitted from the optical coupling module into the switchable reference arm does not fall on a preset reference-arm position corresponding to the specific interface of the eye under test, adjusting an optical path of the reference light by performing a translation mechanism along an optical-axis, so that the reference light can fall on the preset reference-arm position.

Compared to the prior art, the optical biometer and its operating method proposed in this invention can accurately measure the interfaces at different depths in the eye and provide a wider range of optical path modulation, enabling rapid switching between different optical paths simultaneously, so that the optical biometer and its operating method of the invention can overcome manufacturing tolerances and effectively improve the accuracy of measurements from optical biometers.

The advantages and spirit of the present invention can be further understood from the following detailed description of the invention and the accompanying drawings.

### BRIEF DESCRIPTION OF THE DRAWINGS

The accompanying drawings of this invention are described below:

FIG. 1 illustrates a schematic diagram of an optical biometer in an embodiment of the invention.

FIG. 2A and FIG. 2B illustrate schematic diagrams showing that the reference light emitted from the optical coupling module into the switchable reference arm does not fall at the preset reference arm position corresponding to a specific interface of the eye under test.

FIG. 2C illustrates a schematic diagram showing that the reference light emitted from the optical coupling module into the switchable reference arm falls at the preset reference arm position corresponding to a specific interface of the eye under test.

FIG. 3A illustrates a schematic diagram of performing the translation mechanism on the optical coupling module along the optical axis to adjust the optical path of the reference light accordingly.

FIG. 3B illustrates a schematic diagram of performing the translation mechanism on the reference arm along the optical axis to adjust the optical path of the reference light accordingly.

FIG. 3C illustrates a schematic diagram of performing the translation mechanism on the light source module along the optical axis to adjust the optical path of the reference light accordingly.

FIG. 4A and FIG. 4B illustrate schematic diagrams of changing the thickness of the light-transmitting medium along the optical axis to adjust the optical path of the reference light accordingly.

FIG. 5 illustrates a flowchart of the optical biometer operating method in another embodiment of the invention.

FIG. 6 illustrates a flowchart of the optical biometer operating method in another embodiment of the invention.

### DETAILED DESCRIPTION

Reference will now be made in detail to exemplary embodiments of the invention, examples of which are illustrated in the accompanying drawings. Elements/components using the same or similar reference numerals in the drawings and embodiments are used to represent the same or similar parts.

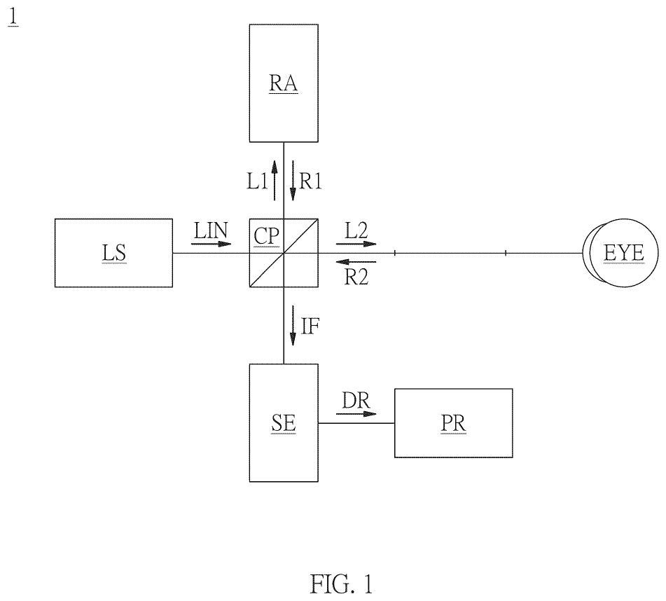

A specific embodiment of the invention is an optical biometer. In this embodiment, the optical biometer can be an optical coherence tomography (OCT) biometer, but not limited t o this. Please refer to FIG. 1, which illustrates a schematic diagram of the optical biometer in this embodiment.

As shown in FIG. 1, the optical biometer 1 includes a light source module LS, an optical coupling module CP, a switchable reference arm RA, a detection module SE and a processing module PR. The optical coupling module CP is disposed between the light source module LS and the eye EYE under test, and also between the switchable reference arm RA and the detection module SE. The detection module SE is coupled to the processing module PR. In practical applications, there are no specific restrictions on the type of light source in the light source module LS; the optical coupling module CP can include a beam splitter, but not limited t o this; the processing module PR can be a microcontroller unit (MCU) or a central processing unit (CPU), but not limited to this.

The light source module LS is configured to emit an incident light LIN to the optical coupling module CP. When the incident light LIN is emitted to the optical coupling module CP, the optical coupling module CP is configured to split the incident light LIN into a reference light L1 and a sensing light L2, and then emit the reference light L1 toward the switchable reference arm RA and emit the sensing light L2 toward the eye EYE under test respectively.

When the reference light L1 is emitted into the switchable reference arm RA, the switchable reference arm RA is configured to reflect the reference light L1 to generate a first reflected light R1 to the optical coupling module CP. When the sensing light L2 is emitted on a specific interface of the eye EYE under test, the specific interface of the eye EYE under test reflects the sensing light L2 to generate a second reflected light R2 to the optical coupling module CP. It should be noted that when the optical biometer 1 operates in a retina mode, the specific interface of the eye EYE under test is retina; when the optical biometer 1 operates in a cornea mode, the specific interface of the eye EYE under test is cornea, but not limited to this.

When the optical coupling module CP receives the first reflected light R1 from the switchable reference arm RA and receives the second reflected light R2 from the eye EYE under test, the optical coupling module CP is configured to cause the first reflected light R1 and the second reflected light R2 to interfere to generate an optical interference signal IF to the detection module SE. The detection module SE is configured to generate a detection result DR for the eye EYE under test based on the optical interference signal IF, and then transmit the detection result DR to the processing module PR. The processing module PR is configured to analyze and process the detection result DR to obtain information such as relative positions of the retina and cornea, and an axial length of the eye EYE under test.

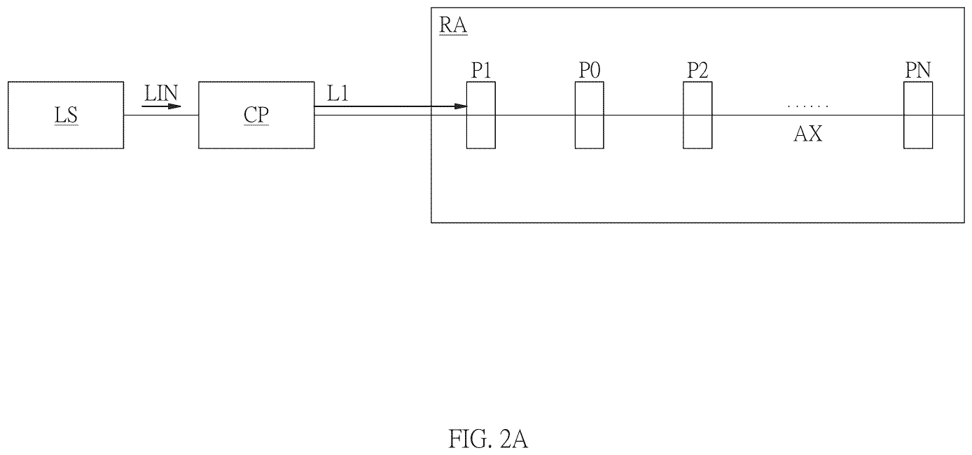

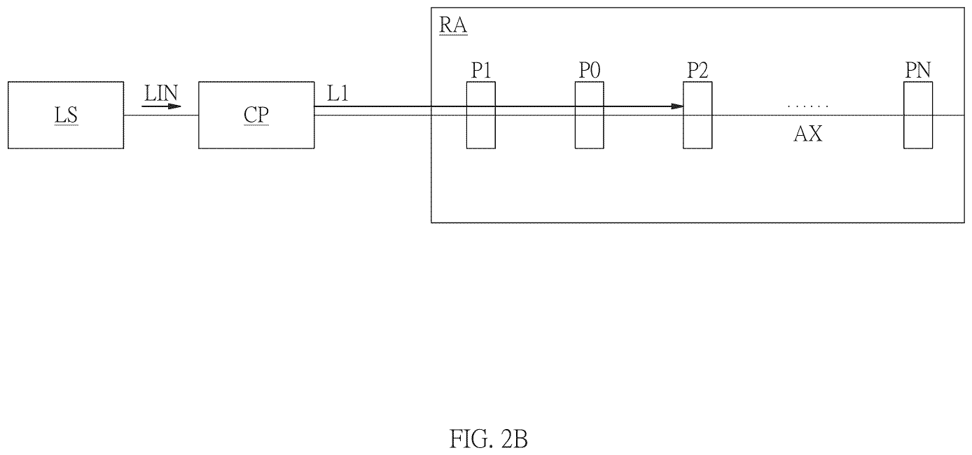

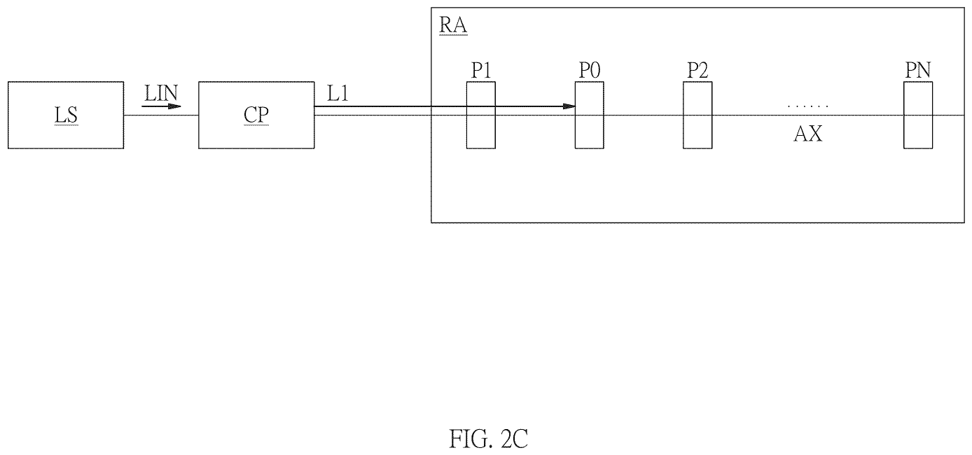

Please refer to FIG. 2A and FIG. 2B, due to the manufacturing tolerances, the reference light L1 emitted from the optical coupling module CP into the switchable reference arm RA may not fall on a preset reference arm position P0 corresponding to the specific interface of the eye EYE under test along an optical axis direction AX. For example, FIG. 2A shows the reference light L1 falling on the reference arm position P1 along the optical axis direction AX, and FIG. 2B shows the reference light L1 falling on the reference arm position P2 along the optical axis direction AX.

Therefore, to overcome this drawback, when the reference light L1 emitted into the switchable reference arm RA does not fall on the preset reference arm position P0, the optical biometer 1 of the invention can perform a translation mechanism in the optical axis direction AX to adjust the optical path of the reference light L1 in a stepped or stepless manner, so that the adjusted reference light L1 can smoothly fall on the preset reference arm position P0 as shown in FIG. 2C.

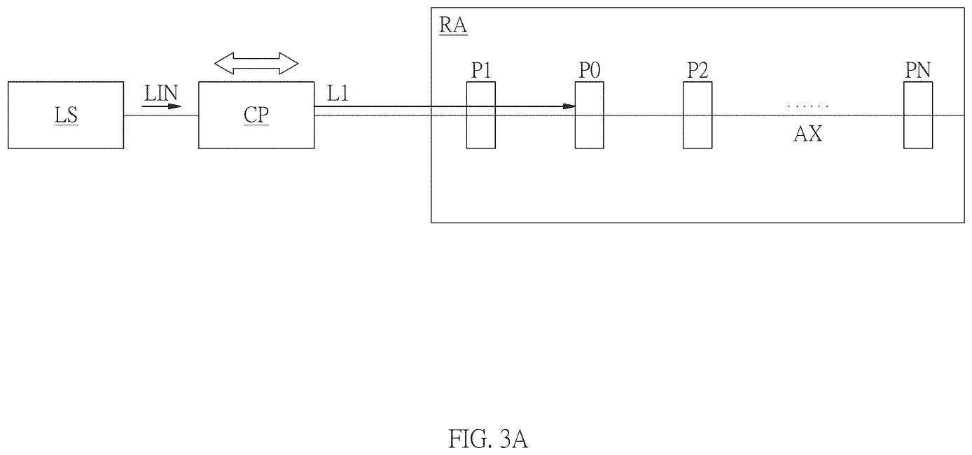

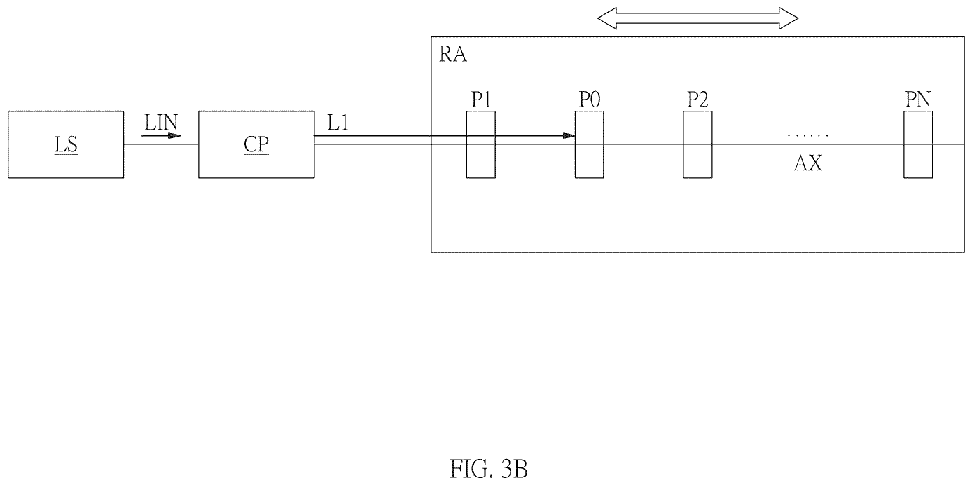

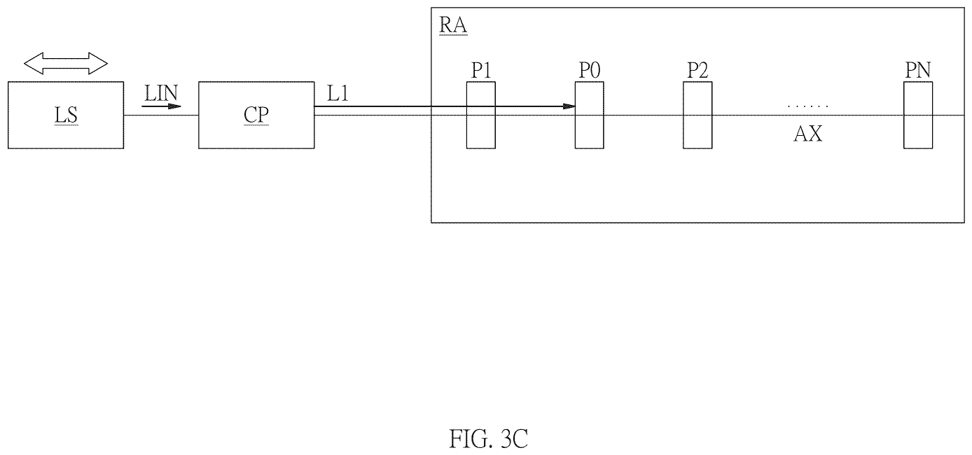

In practical applications, this translation mechanism can include adjusting the position of the optical coupling module CP in the optical axis direction AX as shown in FIG. 3A, adjusting the position of the switchable reference arm RA in the optical axis direction AX as shown in FIG. 3B, or adjusting the position of the light source module LS in the optical axis direction as shown in FIG. 3C, but not limited to this.

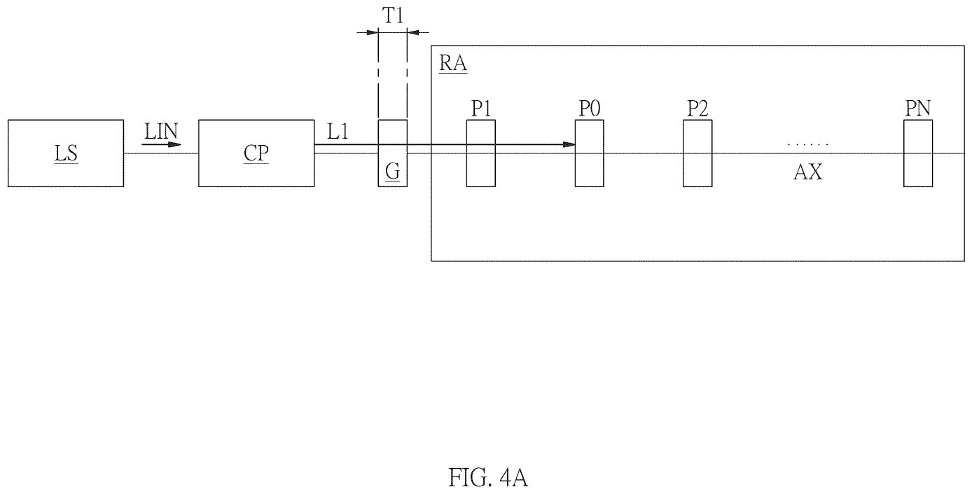

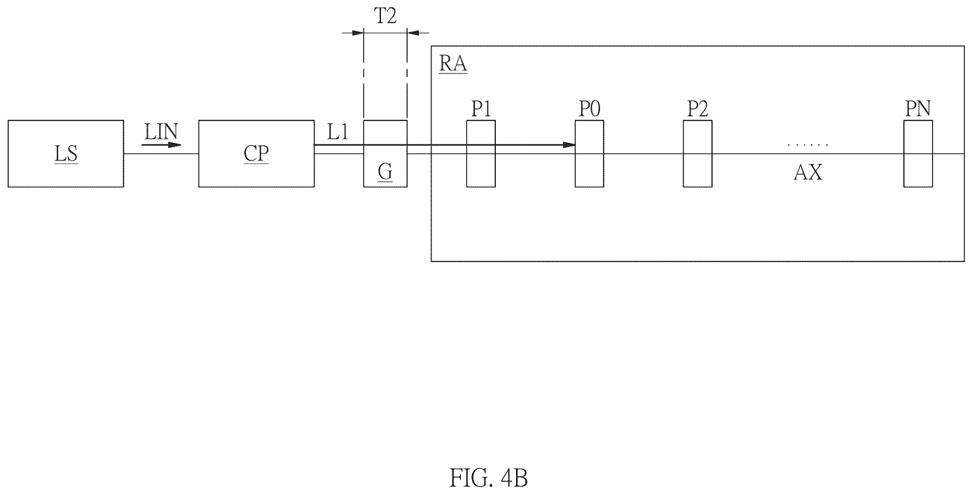

In another embodiment of the invention, please refer to FIG. 4A and FIG. 4B, the optical biometer 1 can further include a light-transmitting medium G disposed between the optical coupling module CP and the switchable reference arm RA. When the reference light L1 emitted into the switchable reference arm RA does not fall on the preset reference arm position P0, the optical biometer 1 of the invention can adjust the optical path of the reference light L1 by changing a thickness of the light-transmitting medium G in the optical axis direction AX, so that the reference light L1 can fall on the preset reference arm position P0.

For example, FIG. 4A shows that the thickness of the light-transmitting medium G is adjusted to a first thickness T1, and FIG. 4B shows that the thickness of the light-transmitting medium G is adjusted to a second thickness T2, wherein the second thickness T2 is greater than the first thickness T1, but not limited to this. In practical applications, the light-transmitting medium G can be glass or acrylic, but not limited to this.

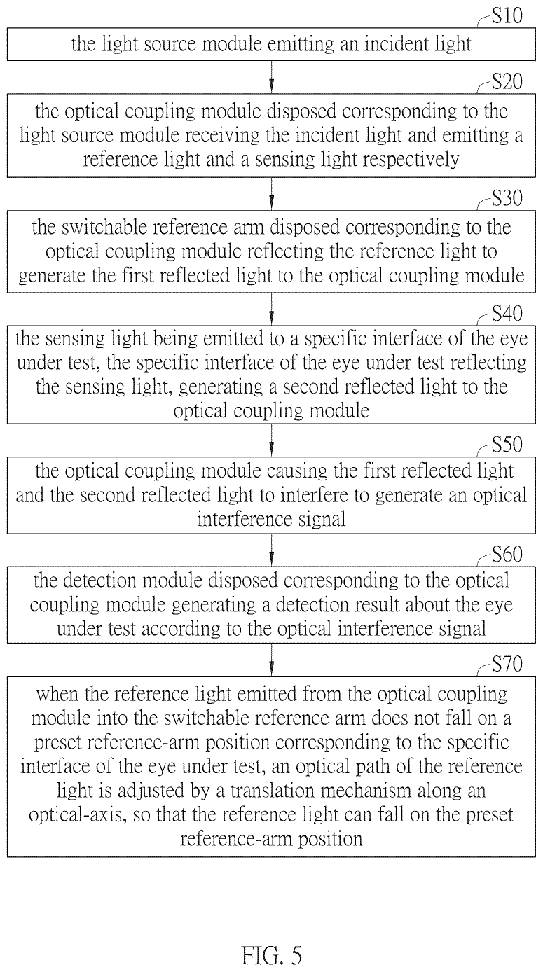

Another specific embodiment of the invention is a method for operating an optical biometer. In this embodiment, the optical biometer includes a light source module, an optical coupling module, a switchable reference arm and a detection module. Please refer to FIG. 5, which illustrates a flowchart of the optical biometer operating method in this embodiment.

As shown in FIG. 5, the optical biometer operating method in this embodiment includes, but not limited to, the following steps of:
Step S10: the light source module emitting an incident light;
Step S20: the optical coupling module disposed corresponding to the light source module receiving the incident light and emitting a reference light and a sensing light respectively;
Step S30: the switchable reference arm disposed corresponding to the optical coupling module reflecting the reference light to generate the first reflected light to the optical coupling module;
Step S40: the sensing light being emitted to a specific interface of the eye under test, the specific interface of the eye under test reflecting the sensing light, generating a second reflected light to the optical coupling module;
Step S50: the optical coupling module causing the first reflected light and the second reflected light to interfere to generate an optical interference signal;
Step S60: the detection module disposed corresponding to the optical coupling module generating a detection result related to the eye under test according to the optical interference signal; and
Step S70: when the reference light emitted from the optical coupling module into the switchable reference arm does not fall on a preset reference-arm position corresponding to the specific interface of the eye under test, an optical path of the reference light is adjusted by a translation mechanism along an optical-axis, so that the reference light can fall on the preset reference-arm position.

In practical applications, when the optical biometer operates in a retinal mode, the specific interface of the eye under test is retina; when the optical biometer operates in a corneal mode, the specific interface of the eye under test is cornea, but not limited to this.

In an embodiment, the method of adjusting the optical path of the reference light by performing a translation mechanism along the optical axis can be a stepped or stepless adjustment, ensuring that the reference light falls on the preset reference arm position. In practice, this translation mechanism can include adjusting the position of the optical coupling module, the switchable reference arm, or the light source module along the optical axis, but not limited to this.

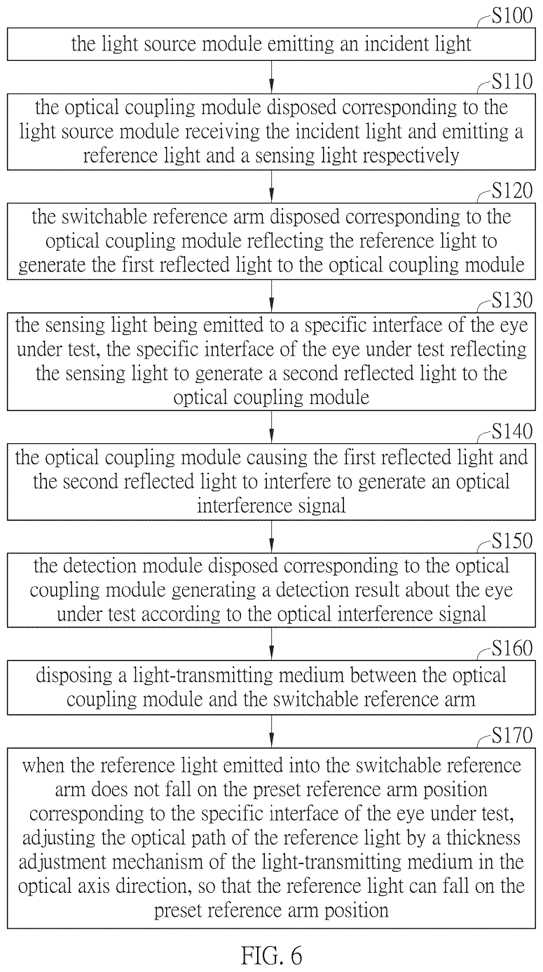

In another embodiment of the invention, as shown in FIG. 6, the optical biometer operating method in this embodiment includes, but not limited to, the following steps of:
Step S100: the light source module emitting an incident light;
Step S110: the optical coupling module disposed corresponding to the light source module receiving the incident light and emitting a reference light and a sensing light respectively;
Step S120: the switchable reference arm disposed corresponding to the optical coupling module reflecting the reference light to generate a first reflected light to the optical coupling module;
Step S130: the sensing light being emitted to a specific interface of the eye under test, the specific interface of the eye under test reflecting the sensing light to generate a second reflected light to the optical coupling module;
Step S140: the optical coupling module causing the first reflected light and the second reflected light to interfere to generate an optical interference signal;
Step S150: the detection module disposed corresponding to the optical coupling module generating a detection result related to the eye under test according to the optical interference signal;
Step S160: disposing a light-transmitting medium between the optical coupling module and the switchable reference arm; and
Step S170: when the reference light emitted into the switchable reference arm does not fall on the preset reference arm position corresponding to the specific interface of the eye under test, adjusting the optical path of the reference light by a thickness adjustment mechanism of the light-transmitting medium in the optical axis direction, so that the reference light can fall on the preset reference arm position.

Compared to the prior art, the optical biometer and its operating method proposed in this invention can accurately measure the interfaces at different depths in the eye and provide a wider range of optical path modulation, enabling rapid switching between different optical paths simultaneously, so that the optical biometer and its operating method of the invention can overcome manufacturing tolerances and effectively improve the accuracy of measurements from optical biometers.

The above-described embodiments are only for the convenience of illustrating the present invention and are not intended to limit it. Various simple modifications and alterations made by those skilled in the art based on the claims and description of the present invention without departing from the spirit and scope of the present invention should still be included in the following claims.

What is claimed is:

1. An optical biometer, comprising:
a light source module, configured to emit an incident light;
an optical coupling module, disposed corresponding to the light source module and configured to receive the incident light and emit a reference light and a sensing light respectively;
a switchable reference arm, disposed corresponding to the optical coupling module and configured to reflect the reference light to generate a first reflected light to the optical coupling module, when the sensing light is emitted to a specific interface of an eye under test, the specific interface of the eye under test reflecting the sensing light to generate a second reflected light to the optical coupling module, the optical coupling module causing the first reflected light and the second reflected light to interfere to generate an optical interference signal; and
a detection module, disposed corresponding to the optical coupling module and configured to generate a detection result related to the eye under test according to the optical interference signal;
wherein when the reference light emitted from the optical coupling module into the switchable reference arm does not fall on a preset reference-arm position corresponding to the specific interface of the eye under test, an optical path of the reference light is adjusted by a translation mechanism along an optical axis, so that the reference light can fall on the preset reference-arm position.

2. The optical biometer according to claim 1, wherein when the optical biometer operates in a retina mode, the specific interface of the eye under test is retina; when the optical biometer operates in a cornea mode, the specific interface of the eye under test is cornea.

3. The optical biometer according to claim 1, wherein the translation mechanism comprises adjusting a position of the optical coupling module, the switchable reference arm, or the light source module along the optical axis.

4. The optical biometer according to claim 1, wherein an adjustment performed on the optical path of the reference light is stepped adjustment or stepless adjustment.

5. An optical biometer, comprising:
a light source module, configured to emit an incident light;
an optical coupling module, disposed corresponding to the light source module and configured to receive the incident light and emit a reference light and a sensing light respectively;
a switchable reference arm, disposed corresponding to the optical coupling module and configured to reflect the reference light to generate a first reflected light to the optical coupling module, when the sensing light is emitted to a specific interface of an eye under test, the specific interface of the eye under test reflecting the sensing light to generate a second reflected light to the optical coupling module, the optical coupling module causing the first reflected light and the second reflected light to interfere to generate an optical interference signal;
a detection module, disposed corresponding to the optical coupling module and configured to generate a detection result related to the eye under test according to the optical interference signal; and
a light-transmitting medium, disposed between the optical coupling module and the switchable reference arm;
wherein when the reference light emitted from the optical coupling module into the switchable reference arm does not fall on a preset reference-arm position corresponding to the specific interface of the eye under test, an optical path of the reference light is adjusted by a translation mechanism along an optical axis, so that the reference light can fall on the preset reference-arm position.

6. A method for operating an optical biometer, the optical biometer comprising a light source module, an optical coupling module, a switchable reference arm and a detection module, the method comprising steps of:
the light source module emitting an incident light;
the optical coupling module disposed corresponding to the light source module receiving the incident light and emitting a reference light and a sensing light respectively;
the switchable reference arm disposed corresponding to the optical coupling module reflecting the reference light to generate a first reflected light to the optical coupling module;
the sensing light being emitted to a specific interface of an eye under test, the specific interface of the eye under test reflecting the sensing light to generate a second reflected light to the optical coupling module;
the optical coupling module causing the first reflected light and the second reflected light to interfere to generate an optical interference signal;
the detection module disposed corresponding to the optical coupling module generating a detection result related to the eye under test according to the optical interference signal; and
when the reference light emitted from the optical coupling module into the switchable reference arm does not fall on a preset reference-arm position corresponding to the specific interface of the eye under test, an optical path of the reference light is adjusted by a translation mechanism along an optical-axis, so that the reference light can fall on the preset reference-arm position.

7. The method according to claim 6, wherein when the optical biometer operates in a retina mode, the specific interface of the eye under test is retina; when the optical biometer operates in a cornea mode, the specific interface of the eye under test is cornea.

8. The method according to claim 6, wherein the translation mechanism comprises adjusting a position of the optical coupling module, the switchable reference arm, or the light source module along the optical axis.

9. The method according to claim 6, wherein an adjustment performed on the optical path of the reference light is stepped adjustment or stepless adjustment.

10. A method for operating an optical biometer, the optical biometer comprising a light source module, an optical coupling module, a switchable reference arm and a detection module, the method comprising steps of:
the light source module emitting an incident light;
the optical coupling module disposed corresponding to the light source module receiving the incident light and emitting a reference light and a sensing light respectively;
the switchable reference arm disposed corresponding to the optical coupling module reflecting the reference light to generate a first reflected light to the optical coupling module;
the sensing light being emitted to a specific interface of an eye under test, the specific interface of the eye under test reflecting the sensing light to generate a second reflected light to the optical coupling module;
the optical coupling module interfering the first reflected light and the second reflected light to generate an optical interference signal;
the detection module disposed corresponding to the optical coupling module generating a detection result related to the eye under test according to the optical interference signal;
disposing a light-transmitting medium between the optical coupling module and the switchable reference arm; and
when the reference light emitted from the optical coupling module into the switchable reference arm does not fall on a preset reference-arm position corresponding to the specific interface of the eye under test, adjusting an optical path of the reference light by performing a translation mechanism along an optical-axis, so that the reference light can fall on the preset reference-arm position.
